(19) United States
(12) Patent Application Publication
(10) Pub. No.: US 2024/0252095 A1
(43) Pub. Date: Aug. 1, 2024

(54) SYSTEM AND METHOD FOR BEAT-BASED ARRHYTHMIA DETECTION

(71) Applicant: Bardy Diagnostics, Inc., Bellevue, WA (US)

(72) Inventors: Ezra Dreisbach, Vashon, WA (US); Rodney Boleyn, Bellevue, WA (US); Noah Herrin, Seattle, WA (US); Aron K. Daw, Bellevue, WA (US)

(21) Appl. No.: 18/418,990

(22) Filed: Jan. 22, 2024

Related U.S. Application Data
(60) Provisional application No. 63/442,038, filed on Jan. 30, 2023.

(51) Int. Cl. A61B 5/364; A61B 5/00; A61B 5/024; A61B 5/308

(52) U.S. Cl. CPC A61B 5/364; A61B 5/0006; A61B 5/002; A61B 5/02438; A61B 5/308; A61B 5/6833

(57) ABSTRACT

The efficient utilization is provided within a cloud-computing environment where a master plane executing on a master node controls spinning up and down worker nodes, with worker nodes whose output is necessary for functioning of other worker nodes being spun up before the worker nodes needing that output. By dedicating the available computational resources to the earlier-spun worker nodes, these worker nodes are able to process a greater amount of monitoring data. Once the output of these worker nodes is ready, the available resources are reallocated to the worker nodes that use the output of the earlier-spun worker nodes. Additional control over the cardiac monitoring can be provided by communicating with a monitor to increase the rate at which the monitor transmits the collected data as well as pausing generation of alerts based on the monitoring data if such alerts are likely to be inaccurate.

### CROSS-REFERENCE TO RELATED APPLICATION

This application claims the benefit of U.S. Provisional App. 63/442,038, filed Jan. 30, 2023, titled SYSTEM AND METHOD FOR BEAT-BASED ARRHYTHMIA DETECTION, the disclosures of which are incorporated by reference herein in their entirety and relied upon.

### FIELD

This application relates in general to electrocardiographic monitoring and processing and, in particular, to a system and method for beat-based arrhythmia detection.

### BACKGROUND

An electrocardiogram (“ECG”) measures and records electrical potentials to visually depict electrical activity of the heart over time. The ECG can be used by physicians to diagnose heart problems and other potential health concerns. ECGs are used in-clinic during appointments, and, as a result, are limited to recording only those heart-related aspects present at the time of recording. Sporadic conditions that may not show up during a spot ECG recording require other means to diagnose them. These disorders include fainting or syncope; rhythm disorders, such as tachyarrhythmias and bradyarrhythmias; apneic episodes; and other cardiac and related disorders. Thus, an ECG only provides a partial picture and can be insufficient for complete patient diagnosis of many cardiac disorders.

Diagnostic efficacy can be improved, when appropriate, through the use of long-term extended ECG monitoring. Recording sufficient ECG and related physiology over an extended period is challenging, and often essential to enabling a physician to identify events of potential concern. Ambulatory monitoring in-clinic is implausible and impracticable. Nevertheless, if a patient's ECG could be recorded in an ambulatory setting, thereby allowing the patient to engage in activities of daily living, the chances of acquiring meaningful information and capturing an abnormal event while the patient is engaged in normal activities becomes more likely to be achieved.

Cardiac and physiological monitors that allow use by a patient outside a medical setting, while providing ambulatory monitoring, exist. Generally, each monitor records cardiac and/or physiological data from a patient over a period up to two weeks and stores the data on flash memory installed on the monitor. The amount of data recorded is limited by the size of the flash memory, which in turn limits the amount of recording time by the monitor. Unfortunately, such a monitor may miss a cardiac event if the event does not occur within the recording period. For example, some types of arrhythmias require long term monitoring because occurrences of the arrhythmia do not show often. Further, long term monitoring can be helpful to monitor the effectiveness of anti-arrhythmia drugs and determine whether any changes need be made to the prescribed dose.

Once the storage is at capacity, the patient removes the monitor and sends the monitor to a data processing center where the data is downloaded and analyzed. Alternatively, the patient can go to a medical facility to download the data. The downloaded data can be processed using software, manual review, or a combination of software and manual review to perform beat detection, event detection, and condition diagnosis. However, waiting until memory on a monitor has reached capacity and offloading the data all at a single time can create a delay in obtaining important information, including whether the patient experienced a cardiac event. Accordingly, such existing devices are limited to an amount of data capable of being stored.

Therefore, a need remains for an extended wear continuously recording cardiac and physiology monitor capable of being worn for long periods of time and capable of transferring data in real time or near real time data to perform data processing for identifying medical events sooner. Preferably, such transfer of data occurs via mobile cardiac outpatient telemetry.

### SUMMARY

Physiological monitoring can be provided through a wearable monitor that includes a flexible extended wear electrode patch and a reusable monitor recorder. The wearable monitor sits centrally (in the midline) on the patient's chest along the sternum oriented top-to-bottom. A wireless transceiver, such as a Bluetooth chip, can either provide data or other information to, or receive data or other information from, an interfacing wearable physiology and activity sensor, or wearable or mobile communications devices for relay to a further device, such as a server, for analysis using mobile cardiac outpatient telemetry.

In light of the disclosure set forth herein, and without limiting the disclosure in any way, in a first aspect of the present disclosure, which may be combined with any other aspect, or portion thereof, a system for beat-based arrhythmia detection is provided. The system includes a database to store cardiac data; a cardiac monitor to continuously transmit the cardiac data collected to the database via a mobile application running on a mobile device associated with a patient; and a server. The server includes a central processing unit, memory, an input port, and an output port.

The central processing unit is configured to identify beats from the cardiac data; run detection for premature ventricular contractions and identify one or more beats as representing premature ventricular contractions; compare at least one of the identified beats with each of a plurality of nearby beats by lining up the nearby beat and the identified beat; determine an area between the identified beat and each nearby beat; and mark at least one of the nearby beats as representing premature ventricular contraction when the area between that nearby beat and the identified beat satisfies a threshold level of similarity.

In a second aspect of the present disclosure, which may be combined with any other aspect, or portion thereof, the cardiac data is transmitted from the cardiac monitor using mobile cardiac telemetry.

In a third aspect of the present disclosure, which may be combined with any other aspect, or portion thereof, the cardiac monitor includes a cutaneous monitor placed on skin of the patient or an implantable monitor.

In a fourth aspect of the present disclosure, which may be combined with any other aspect, or portion thereof, the cardiac data is transmitted from the cardiac monitor via a wireless transceiver.

In a fifth aspect of the present disclosure, which may be combined with any other aspect, or portion thereof, the wireless transceiver includes a Bluetooth chip.

In a sixth aspect of the present disclosure, which may be combined with any other aspect, or portion thereof, the cardiac monitor further includes an electrocardiography patch, an externally-powered micro-controller, and an electrocardiographic front end circuit electrically interfaced to the micro-controller and operable to sense electrocardiographic signals as the cardiac data.

In a seventh aspect of the present disclosure, which may be combined with any other aspect, or portion thereof, the nearby beat and the identified beat are lined up based on one of start time and stop time, and shape.

In an eighth aspect of the present disclosure, which may be combined with any other aspect, or portion thereof, the detection for premature ventricular contractions is performed by establishing an average heart rate of the patient by computing all beat times for heart beats over narrow windows of heart beat data, comparing individual heart beats with the average heart rate, and identifying any individual heart beats that occur earlier than the average as premature.

In a ninth aspect of the present disclosure, which may be combined with any other aspect, or portion thereof, the central processing unit performs arrhythmia detection on the premature ventricular contraction beats and the cardiac data.

In a tenth aspect of the present disclosure, which may be combined with any other aspect, or portion thereof, the central processing unit flags one or more segments of the cardiac data as representing a cardiac condition based on the arrhythmia detection.

In an eleventh aspect of the present disclosure, which may be combined with any other aspect, or portion thereof, a method for beat-based arrhythmia detection is provided. The method include continuously transmitting cardiac data from a cardiac monitor worn by a patient to storage via a mobile application running on a mobile device associated with the patient; identifying beats from the cardiac data; running detection for premature ventricular contractions and identifying one or more beats as representing premature ventricular contractions; comparing at least one of the identified beats with each of a plurality of nearby beats by lining up the nearby beat and the identified beat; determining an area between the identified beat and each nearby beat; and marking at least one of the nearby beats as representing premature ventricular contraction when the area between that nearby beat and the identified beat satisfies a threshold level of similarity.

In a twelfth aspect of the present disclosure, which may be combined with any other aspect, or portion thereof, the cardiac data is transmitted from the cardiac monitor using mobile cardiac telemetry.

In a thirteenth aspect of the present disclosure, which may be combined with any other aspect, or portion thereof, the cardiac monitor includes a cutaneous monitor placed on skin of the patient or an implantable monitor.

In a fourteenth aspect of the present disclosure, which may be combined with any other aspect, or portion thereof, the method further includes transmitting the cardiac data from the cardiac monitor via a wireless transceiver.

In a fifteenth aspect of the present disclosure, which may be combined with any other aspect, or portion thereof, the wireless transceiver includes a Bluetooth chip.

In a sixteenth aspect of the present disclosure, which may be combined with any other aspect, or portion thereof, the cardiac monitor further includes an electrocardiography patch, an externally-powered micro-controller, and an electrocardiographic front end circuit electrically interfaced to the micro-controller and operable to sense electrocardiographic signals as the cardiac data.

In a seventeenth aspect of the present disclosure, which may be combined with any other aspect, or portion thereof, the nearby beat and the identified beat are lined up based on one of start time and stop time, and curves.

In an eighteenth aspect of the present disclosure, which may be combined with any other aspect, or portion thereof, the method further includes establishing an average heart rate of the patient by computing all beat times for heart beats over narrow windows of heart beat data; and comparing individual heart beats with the average heart rate; and identifying any individual heart beats that occur earlier than the average as premature.

In a nineteenth aspect of the present disclosure, which may be combined with any other aspect, or portion thereof, the method further includes performing arrhythmia detection on the premature ventricular contraction beats and the cardiac data

In a twentieth aspect of the present disclosure, which may be combined with any other aspect, or portion thereof, the method further includes flagging one or more segments of the cardiac data as representing a cardiac condition based on the arrhythmia detection.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment is not required to have all of the advantages listed herein. Moreover, it should be noted that the language used in the specification has been selected for readability and instructional purposes, and not to limit the scope of the present subject matter.

### BRIEF DESCRIPTION OF THE DRAWINGS

Understanding that figures depict only typical embodiments of the invention and are not to be considered to limit the scope of the present disclosure, the present disclosure is described and explained with additional specificity and detail through the use of the accompanying figures. The figures are listed below.

FIGS. 1 and 2 are diagrams showing, by way of examples, an extended wear electrocardiography and physiological sensor monitor respectively fitted to the sternal region of a female patient and a male patient.

FIG. 3 is a functional block diagram showing a system for remote interfacing of an extended wear electrocardiography and physiological sensor monitor in accordance with one embodiment.

FIG. 4 is a perspective view showing an extended wear electrode patch with a monitor recorder inserted.

FIG. 5 is a perspective view showing the monitor recorder of FIG. 4.

FIG. 6 is a perspective view showing the extended wear electrode patch of FIG. 4 without a monitor recorder inserted.

FIG. 7 is a bottom plan view of the monitor recorder of FIG. 4.

FIG. 8 is atop view showing the flexible circuit of the extended wear electrode patch of FIG. 4 when mounted above the flexible backing.

FIG. 9 is a functional block diagram showing the component architecture of the circuitry of the monitor recorder of FIG. 4.

FIG. 10 is a functional block diagram showing the circuitry of the extended wear electrode patch of FIG. 4.

FIG. 11 is a flow diagram showing a monitor recorder-implemented method for monitoring ECG data for use in the monitor recorder of FIG. 4.

FIG. 12 is a graph showing, by way of example, a typical ECG waveform.

FIG. 13 is a flow diagram showing a method for offloading and converting ECG and other physiological data from an extended wear electrocardiography and physiological sensor monitor in accordance with one embodiment.

FIG. 14 is a flow diagram showing, by way of example, a method for beat-based arrhythmia detection.

FIG. 15 is a flow diagram showing, by way of example, a method 170 for identifying PVC beats.

FIGS. 16A-D each represent a graph of a PVC beat and non-PVC beat.

FIG. 17 is a block diagram showing, by way of example, different arrhythmia conditions.

### DETAILED DESCRIPTION

Example embodiments will now be described more fully with reference to the accompanying drawings. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specific the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent”). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

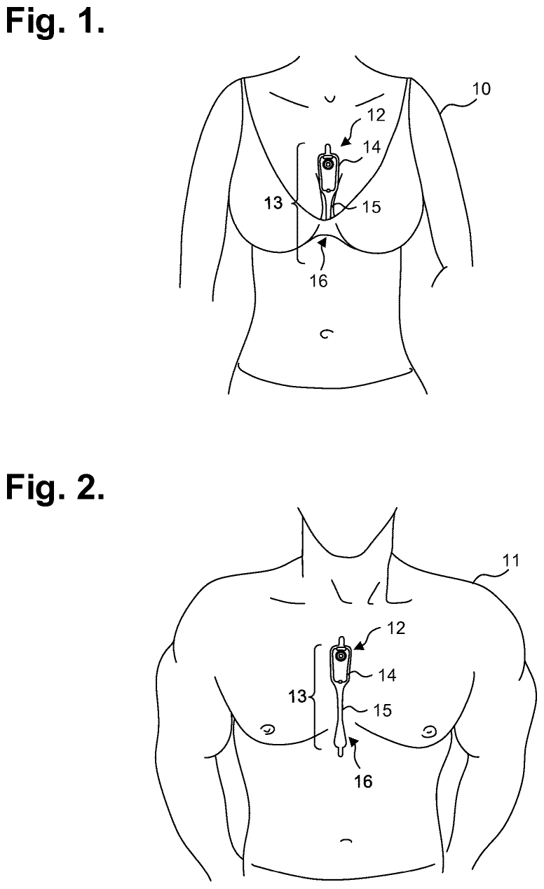

Physiological monitoring can be provided through a wearable monitor that includes two components, a flexible extended wear electrode patch and a removable reusable monitor recorder. FIGS. 1 and 2 are diagrams showing, by way of examples, an extended wear electrocardiography and physiological sensor monitor 12, including a monitor recorder 14 in accordance with one embodiment, respectively fitted to the sternal region of a female patient 10 and a male patient 11. The wearable monitor 12 sits centrally (in the midline) on the patient's chest along the sternum 13 oriented top-to-bottom with the monitor recorder 14 preferably situated towards the patient's head. In a further embodiment, the orientation of the wearable monitor 12 can be corrected post-monitoring, as further described infra. The electrode patch 15 is shaped to fit comfortably and conformal to the contours of the patient's chest approximately centered on the sternal midline 16 (or immediately to either side of the sternum 13). The distal end of the electrode patch 15 extends towards the Xiphoid process and, depending upon the patient's build, may straddle the region over the Xiphoid process. The proximal end of the electrode patch 15, located under the monitor recorder 14, is below the manubrium and, depending upon patient's build, may straddle the region over the manubrium.

The placement of the wearable monitor 12 in a location at the sternal midline 16 (or immediately to either side of the sternum 13) significantly improves the ability of the wearable monitor 12 to cutaneously sense cardiac electric signals, particularly the P-wave (or atrial activity) and, to a lesser extent, the QRS interval signals in the ECG waveforms that indicate ventricular activity, while simultaneously facilitating comfortable long-term wear for many weeks. The sternum 13 overlies the right atrium of the heart and the placement of the wearable monitor 12 in the region of the sternal midline 13 puts the ECG electrodes of the electrode patch 15 in a location better adapted to sensing and recording P-wave signals than other placement locations, say, the upper left pectoral region or lateral thoracic region or the limb leads. In addition, placing the lower or inferior pole (ECG electrode) of the electrode patch 15 over (or near) the Xiphoid process facilitates sensing of ventricular activity and provides superior recordation of the QRS interval.

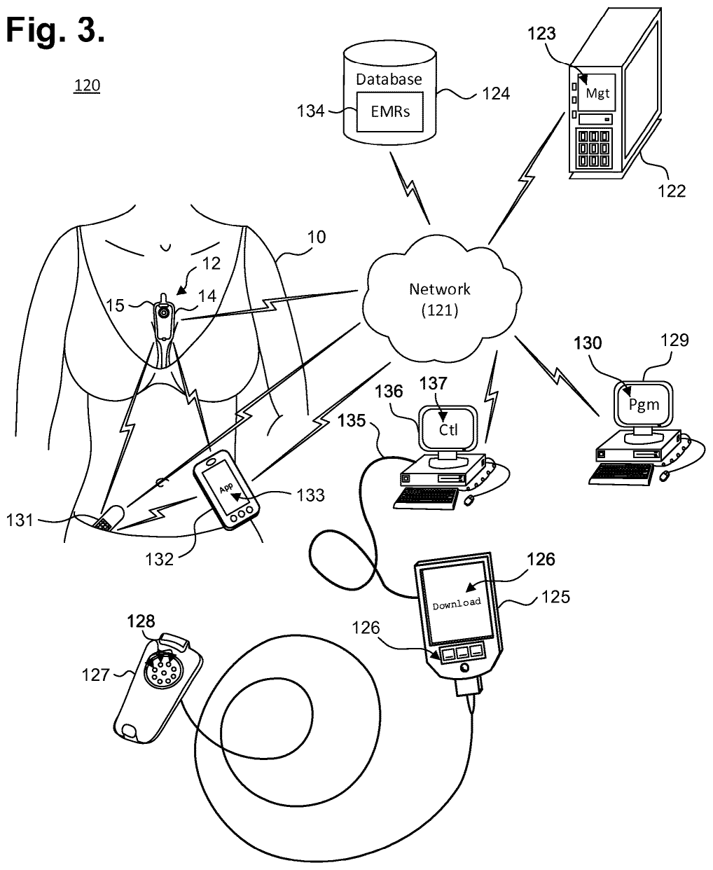

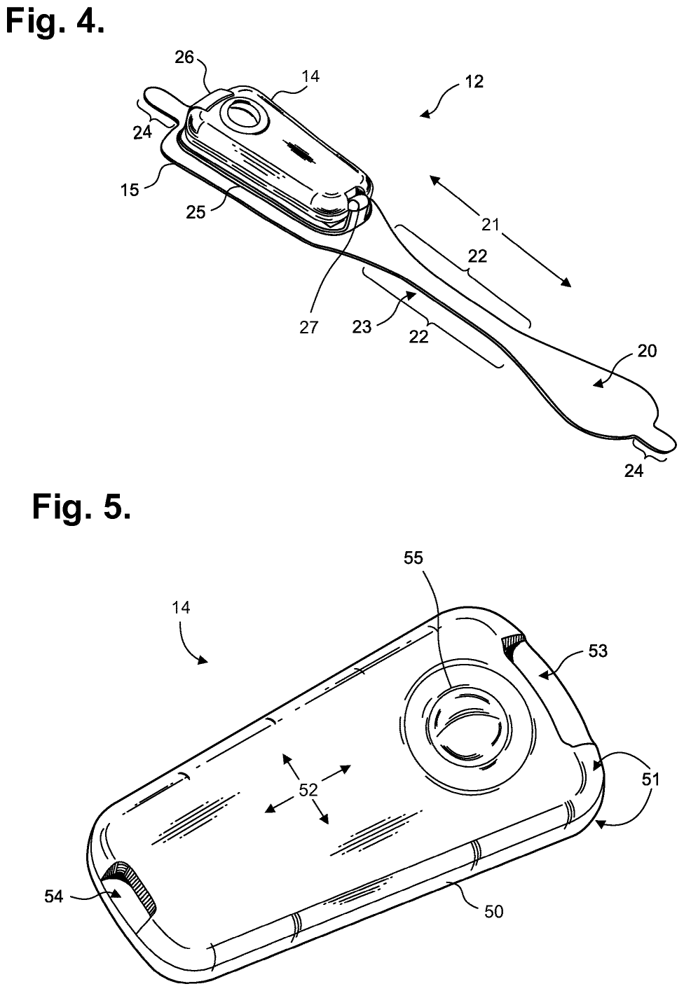

The monitor recorder 14 of the extended wear electrocardiography and physiological sensor monitor 12 senses and records the patient's ECG data into an onboard memory or transmits the data via a wireless transceiver on the monitor 12, which is described further below with respect to FIGS. 9 and 10. The ECG data can include electrical activity, which is used to determine heart beats. In addition, the wearable monitor 12 can interoperate with other devices. FIG. 3 is a functional block diagram showing a system 120 for remote interfacing of an extended wear electrocardiography and physiological sensor monitor 12 in accordance with one embodiment. The monitor recorder 14 is a reusable component that can be fitted during patient monitoring into a non-conductive receptacle provided on the electrode patch 15, as further described infra with reference to FIG. 4, and later removed for offloading of stored ECG data or to receive revised programming. The monitor recorder 14 can then be connected to a download station 125, which could be a programmer or other device that permits the retrieval of stored ECG monitoring data, execution of diagnostics on or programming of the monitor recorder 14, or performance of other functions. The monitor recorder 14 has a set of electrical contacts (not shown) that enable the monitor recorder 14 to physically interface to a set of terminals 128 on a paired receptacle 127 of the download station 125. In turn, the download station 125 executes a communications or offload program 126 (“Offload”) or similar program that interacts with the monitor recorder 14 via the physical interface to retrieve the stored ECG monitoring data. The download station 125 could be a server, personal computer, tablet or handheld computer, smart mobile device, or purpose-built programmer designed specific to the task of interfacing with a monitor recorder 14. Still other forms of download station 125 are possible.

In lieu offloading the ECG data using a download station 125, the ECG data can be offloaded wirelessly via a wireless transceiver, such as a Bluetooth chip, located on the monitor recorder 14 or the electrode patch 15 of the monitor 12. In one embodiment, the patient can download a computer application 132 on their cellular phone 133 or device, which receives the data transmitted wirelessly. Subsequently, the computer application 132 can send the data to the cloud for storing in an electronic medical record (“EMR”) 134 for the patient. The data can also be utilized via the cloud for processing, including beat detection, event detection, diagnosis, and report generation, such as by an independent diagnostic facility.

In addition to the ECG data, the monitor 12 can also record presses of the feedback button provided on the monitor recorder 14. The button press can act as a flag of recorded ECG data before, during, or after that button press. The button press data can be offloaded with the ECG data wirelessly or via a download station.

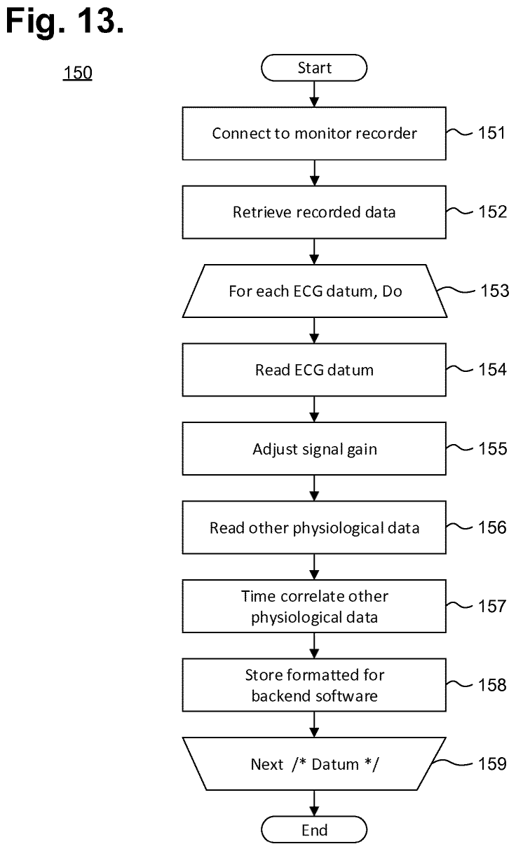

Upon retrieving stored ECG monitoring data from a monitor recorder 14, middleware first operates on the retrieved data to adjust the ECG capture quality, as necessary, and to convert the retrieved data into a format suitable for use by third party post-monitoring analysis software, as further described infra with reference to FIG. 13. The formatted data can then be retrieved from the download station 125 over a hard link 135 using a control program 137 (“Ctl”) or analogous application executing on a personal computer 136 or other connectable computing device, via a communications link (not shown), whether wired or wireless, or by physical transfer of storage media (not shown). The personal computer 136 or other connectable device may also execute middleware that converts ECG data and other information into a format suitable for use by a third-party post-monitoring analysis program, as further described infra with reference to FIG. 13. Note that formatted data stored on the personal computer 136 would have to be maintained and safeguarded in the same manner as the electronic medical records 134 in the secure database 124, as further discussed infra. In a further embodiment, the download station 125 is able to directly interface with other devices over a computer communications network 121, which could be some combination of a local area network and a wide area network, including the Internet, over a wired or wireless connection.

A client-server model could be used to employ a server 122 to remotely interface with the download station 125 over the network 121 and retrieve the formatted data or other information. The server 122 executes a patient management program 123 (“Mgt”) or similar application that stores the retrieved formatted data and other information in a secure database 124 cataloged in that patient's EMRs 134. In addition, the patient management program 123 could manage a subscription service that authorizes a monitor recorder 14 to operate for a set period of time or under pre-defined operational parameters.

The patient management program 123, or other trusted application, also maintains and safeguards the secure database 124 to limit access to patient EMRs 134 to only authorized parties for appropriate medical or other uses, such as mandated by state or federal law, such as under the Health Insurance Portability and Accountability Act (“HIPAA”) or per the European Union's Data Protection Directive. For example, a physician may seek to review and evaluate his patient's ECG monitoring data, as securely stored in the secure database 124. The physician would execute an application program 130 (“Pgm”), such as a post-monitoring ECG analysis program, on a personal computer 129 or other connectable computing device, and, through the application 130, coordinate access to his patient's EMRs 134 with the patient management program 123. Other schemes and safeguards to protect and maintain the integrity of patient EMRs 134 are possible.

The wearable monitor 12 can interoperate wirelessly with other wearable physiology and activity sensors 131 and with wearable or mobile communications devices 133. Wearable physiology and activity sensors 131 encompass a wide range of wirelessly interconnectable devices that measure or monitor data physical to the patient's body, such as heart rate, temperature, blood pressure, and so forth; physical states, such as movement, sleep, footsteps, and the like; and performance, including calories burned or estimated blood glucose level. These devices originate both within the medical community to sense and record traditional medical physiology that could be useful to a physician in arriving at a patient diagnosis or clinical trajectory, as well as from outside the medical community, from, for instance, sports or lifestyle product companies who seek to educate and assist individuals with self-quantifying interests.

Frequently, wearable physiology and activity sensors 131 are capable of wireless interfacing with wearable or mobile communications devices 133, particularly smart mobile devices, including so-called “smart phones,” to download monitoring data either in real-time or in batches. The wearable or mobile communications device 133 executes an application (“App”) that can retrieve the data collected by the wearable physiology and activity sensor 131 and evaluate the data to generate information of interest to the wearer, such as an estimation of the effectiveness of the wearer's exercise efforts. Still other wearable or mobile communications device 133 functions on the collected data are possible.

The wearable or mobile communications devices 133 could also serve as a conduit for providing the data collected by the wearable physiology and activity sensor 131 to a server 122, or, similarly, the wearable physiology and activity sensor 131 could itself directly provide the collected data to the server 122. The server 122 could then merge the collected data into the wearer's EMRs 134 in the secure database 124, if appropriate (and permissible), or the server 122 could perform an analysis of the collected data, perhaps based by comparison to a population of like wearers of the wearable physiology and activity sensor 131. Still other server 122 functions on the collected data are possible.

Finally, the monitor recorder 14 can also be equipped with a wireless transceiver, as further described infra with reference to FIGS. 9 and 10. Thus, when wireless-enabled, both wearable physiology and activity sensors 131 and wearable or mobile communications devices 133 could wirelessly interface with the monitor recorder 14, which could either provide data or other information to, or receive data or other information from an interfacing device for relay to a further device, such as the server 122, analysis, or other purpose. In addition, the monitor recorder 14 could wirelessly interface directly with the server 122, personal computer 129, or other computing device connectable over the network 121, when the monitor recorder 14 is appropriately equipped for interfacing with such devices. Still other types of remote interfacing of the monitor recorder 14 are possible.

During use, the electrode patch 15 is first adhesed to the skin along the sternal midline 16 (or immediately to either side of the sternum 13). A monitor recorder 14 is then snapped into place on the electrode patch 15 to initiate ECG monitoring. FIG. 4 is a perspective view showing an extended wear electrode patch 15 with a monitor recorder 14 in accordance with one embodiment inserted. The body of the electrode patch 15 is preferably constructed using a flexible backing 20 formed as an elongated strip 21 of wrap knit or similar stretchable material with a narrow longitudinal mid-section 23 evenly tapering inward from both sides. A pair of cut-outs 22 between the distal and proximal ends of the electrode patch 15 create a narrow longitudinal midsection 23 or “isthmus” and defines an elongated “hourglass”-like shape, when viewed from above.

The electrode patch 15 incorporates features that significantly improve wearability, performance, and patient comfort throughout an extended monitoring period. During wear, the electrode patch 15 is susceptible to pushing, pulling, and torqueing movements, including compressional and torsional forces when the patient bends forward, and tensile and torsional forces when the patient leans backwards. To counter these stress forces, the electrode patch 15 incorporates strain and crimp reliefs, such as described in commonly-assigned U.S. Pat. No. 9,545,204, entitled “Extended Wear Electrocardiography Patch,” issued Jan. 17, 2017, the disclosure of which is incorporated by reference. In addition, the cut-outs 22 and longitudinal midsection 23 help minimize interference with and discomfort to breast tissue, particularly in women (and gynecomastic men). The cut-outs 22 and longitudinal midsection 23 further allow better conformity of the electrode patch 15 to sternal bowing and to the narrow isthmus of flat skin that can occur along the bottom of the intermammary cleft between the breasts, especially in buxom women. The cut-outs 22 and longitudinal midsection 23 help the electrode patch 15 fit nicely between a pair of female breasts in the intermammary cleft. Still other shapes, cut-outs and conformities to the electrode patch 15 are possible.

The monitor recorder 14 removably and reusably snaps into an electrically non-conductive receptacle 25 during use. The monitor recorder 14 contains electronic circuitry for recording and storing the patient's electrocardiography as sensed via a pair of ECG electrodes provided on the electrode patch 15, such as described in commonly-assigned U.S. Pat. No. 9,730,593, entitled “Extended Wear Ambulatory Electrocardiography and Physiological Sensor Monitor,” issued Aug. 15, 2017, the disclosure which is incorporated by reference. The non-conductive receptacle 25 is provided on the top surface of the flexible backing 20 with a retention catch 26 and tension clip 27 molded into the non-conductive receptacle 25 to conformably receive and securely hold the monitor recorder 14 in place.

The monitor recorder 14 includes a sealed housing that snaps into place in the non-conductive receptacle 25. FIG. 5 is a perspective view showing the monitor recorder 14 of FIG. 4. The sealed housing 50 of the monitor recorder 14 intentionally has a rounded isosceles trapezoidal-like shape 52, when viewed from above, such as described in commonly-assigned U.S. Design U.S. Pat. No. 7,179,955, entitled “Electrocardiography Monitor,” issued Nov. 18, 2014, the disclosure of which is incorporated by reference. The edges 51 along the top and bottom surfaces are rounded for patient comfort. The sealed housing 50 is approximately 47 mm long, 23 mm wide at the widest point, and 7 mm high, excluding a patient-operable tactile-feedback button 55. The sealed housing 50 can be molded out of polycarbonate, ABS, or an alloy of those two materials. The button 55 is waterproof and the button's top outer surface is molded silicon rubber or similar soft pliable material. A retention detent 53 and tension detent 54 are molded along the edges of the top surface of the housing 50 to respectively engage the retention catch 26 and the tension clip 27 molded into non-conductive receptacle 25. Other shapes, features, and conformities of the sealed housing 50 are possible.

The electrode patch 15 is intended to be disposable. The monitor recorder 14, however, is reusable and can be transferred to successive electrode patches 15 to ensure continuity of monitoring. The placement of the wearable monitor 12 in a location at the sternal midline 16 (or immediately to either side of the sternum 13) benefits long-term extended wear by removing the requirement that ECG electrodes be continually placed in the same spots on the skin throughout the monitoring period. Instead, the patient is free to place an electrode patch 15 anywhere within the general region of the sternum 13.

As a result, at any point during ECG monitoring, the patient's skin is able to recover from the wearing of an electrode patch 15, which increases patient comfort and satisfaction, while the monitor recorder 14 ensures ECG monitoring continuity with minimal effort. A monitor recorder 14 is merely unsnapped from a worn out electrode patch 15, the worn out electrode patch 15 is removed from the skin, a new electrode patch 15 is adhered to the skin, possibly in a new spot immediately adjacent to the earlier location, and the same monitor recorder 14 is snapped into the new electrode patch 15 to reinitiate and continue the ECG monitoring.

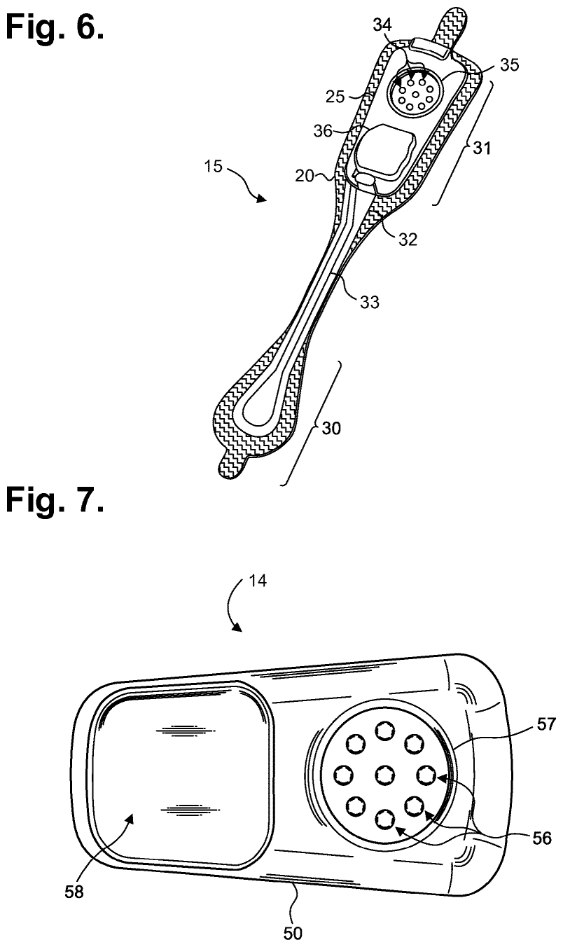

During use, the electrode patch 15 is first adhered to the skin in the sternal region. FIG. 6 is a perspective view showing the extended wear electrode patch 15 of FIG. 4 without a monitor recorder 14 inserted. A flexible circuit 32 is adhered to each end of the flexible backing 20. A distal circuit trace 33 and a proximal circuit trace (not shown) electrically couple ECG electrodes (not shown) to a pair of electrical pads 34. The electrical pads 34 are provided within a moisture-resistant seal 35 formed on the bottom surface of the non-conductive receptacle 25. When the monitor recorder 14 is securely received into the non-conductive receptacle 25, that is, snapped into place, the electrical pads 34 interface to electrical contacts (not shown) protruding from the bottom surface of the monitor recorder 14, and the moisture-resistant seal 35 enables the monitor recorder 14 to be worn at all times, even during bathing or other activities that could expose the monitor recorder 14 to moisture.

In addition, a battery compartment 36 is formed on the bottom surface of the non-conductive receptacle 25, and a pair of battery leads (not shown) electrically interface the battery to another pair of the electrical pads 34. The battery contained within the battery compartment 35 can be replaceable, rechargeable or disposable.

The monitor recorder 14 draws power externally from the battery provided in the non-conductive receptacle 25, thereby uniquely obviating the need for the monitor recorder 14 to carry a dedicated power source. FIG. 7 is a bottom plan view of the monitor recorder 14 of FIG. 4. A cavity 58 is formed on the bottom surface of the sealed housing 50 to accommodate the upward projection of the battery compartment 36 from the bottom surface of the non-conductive receptacle 25, when the monitor recorder 14 is secured in place on the non-conductive receptacle 25. A set of electrical contacts 56 protrude from the bottom surface of the sealed housing 50 and are arranged in alignment with the electrical pads 34 provided on the bottom surface of the non-conductive receptacle 25 to establish electrical connections between the electrode patch 15 and the monitor recorder 14. In addition, a seal coupling 57 circumferentially surrounds the set of electrical contacts 56 and securely mates with the moisture-resistant seal 35 formed on the bottom surface of the non-conductive receptacle 25.

The placement of the flexible backing 20 on the sternal midline 16 (or immediately to either side of the sternum 13) also helps to minimize the side-to-side movement of the wearable monitor 12 in the left- and right-handed directions during wear. To counter the dislodgment of the flexible backing 20 due to compressional and torsional forces, a layer of non-irritating adhesive, such as hydrocolloid, is provided at least partially on the underside, or contact, surface of the flexible backing 20, but only on the distal end 30 and the proximal end 31. As a result, the underside, or contact surface of the longitudinal midsection 23 does not have an adhesive layer and remains free to move relative to the skin. Thus, the longitudinal midsection 23 forms a crimp relief that respectively facilitates compression and twisting of the flexible backing 20 in response to compressional and torsional forces. Other forms of flexible backing crimp reliefs are possible.

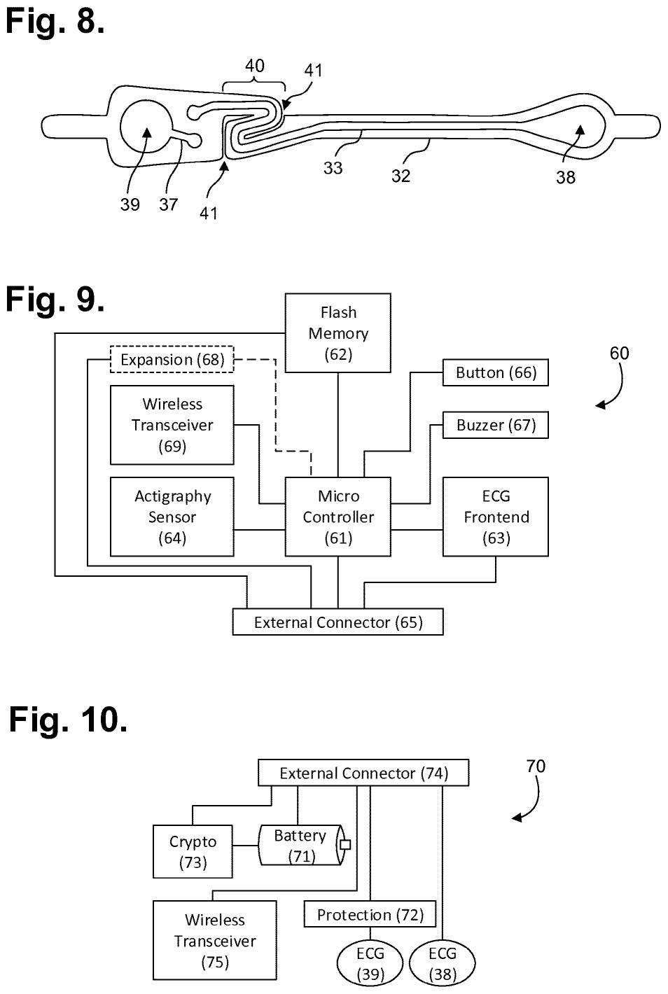

Unlike the flexible backing 20, the flexible circuit 32 is only able to bend and cannot stretch in a planar direction. The flexible circuit 32 can be provided either above or below the flexible backing 20. FIG. 8 is a top view showing the flexible circuit 32 of the extended wear electrode patch 15 of FIG. 4 when mounted above the flexible backing 20. A distal ECG electrode 38 and proximal ECG electrode 39 are respectively coupled to the distal and proximal ends of the flexible circuit 32. A strain relief 40 is defined in the flexible circuit 32 at a location that is partially underneath the battery compartment 36 when the flexible circuit 32 is affixed to the flexible backing 20. The strain relief 40 is laterally extendable to counter dislodgment of the ECG electrodes 38, 39 due to tensile and torsional forces. A pair of strain relief cutouts 41 partially extend transversely from each opposite side of the flexible circuit 32 and continue longitudinally towards each other to define in ‘S’-shaped pattern, when viewed from above. The strain relief respectively facilitates longitudinal extension and twisting of the flexible circuit 32 in response to tensile and torsional forces. Other forms of circuit board strain relief are possible.

ECG monitoring and other functions performed by the monitor recorder 14 are provided through a micro controlled architecture. FIG. 9 is a functional block diagram showing the component architecture of the circuitry 60 of the monitor recorder 14 of FIG. 4. The circuitry 60 is externally powered through a battery provided in the non-conductive receptacle 25 (shown in FIG. 6). Both power and raw ECG signals, which originate in the pair of ECG electrodes 38, 39 (shown in FIG. 8) on the distal and proximal ends of the electrode patch 15, are received through an external connector 65 that mates with a corresponding physical connector on the electrode patch 15. The external connector 65 includes the set of electrical contacts 56 that protrude from the bottom surface of the sealed housing 50 and which physically and electrically interface with the set of pads 34 provided on the bottom surface of the non-conductive receptacle 25. The external connector includes electrical contacts 56 for data download, microcontroller communications, power, analog inputs, and a peripheral expansion port. The arrangement of the pins on the electrical connector 65 of the monitor recorder 14 and the device into which the monitor recorder 14 is attached, whether an electrode patch 15 or download station (not shown), follow the same electrical pin assignment convention to facilitate interoperability. The external connector 65 also serves as a physical interface to a download station that permits the retrieval of stored ECG monitoring data, communication with the monitor recorder 14, and performance of other functions.

Operation of the circuitry 60 of the monitor recorder 14 is managed by a microcontroller 61. The micro-controller 61 includes a program memory unit containing internal flash memory that is readable and writeable. The internal flash memory can also be programmed externally. The micro-controller 61 draws power externally from the battery provided on the electrode patch 15 via a pair of the electrical contacts 56. The microcontroller 61 connects to the ECG front end circuit 63 that measures raw cutaneous electrical signals and generates an analog ECG signal representative of the electrical activity of the patient's heart over time.

The circuitry 60 of the monitor recorder 14 also includes a flash memory 62, which the micro-controller 61 uses for storing ECG monitoring data and other physiology and information. The flash memory 62 also draws power externally from the battery provided on the electrode patch 15 via a pair of the electrical contacts 56. Data is stored in a serial flash memory circuit, which supports read, erase and program operations over a communications bus. The flash memory 62 enables the microcontroller 61 to store digitized ECG data. The communications bus further enables the flash memory 62 to be directly accessed externally over the external connector 65 when the monitor recorder 14 is interfaced to a download station.

The circuitry 60 of the monitor recorder 14 further includes an actigraphy sensor 64 implemented as a 3-axis accelerometer. The accelerometer may be configured to generate interrupt signals to the microcontroller 61 by independent initial wake up and free fall events, as well as by device position. In addition, the actigraphy provided by the accelerometer can be used during post-monitoring analysis to correct the orientation of the monitor recorder 14 if, for instance, the monitor recorder 14 has been inadvertently installed upside down, that is, with the monitor recorder 14 oriented on the electrode patch 15 towards the patient's feet, as well as for other event occurrence analyses, such as described in commonly assigned U.S. Pat. No. 9,737,224, entitled “Event Alerting Through Actigraphy Embedded within Electrocardiographic Data,” issued Aug. 22, 2017, the disclosure of which is incorporated by reference.

The circuitry 60 of the monitor recorder 14 includes a wireless transceiver 69 that can provide wireless interfacing capabilities. The wireless transceiver 69 also draws power externally from the battery provided on the electrode patch 15 via a pair of the electrical contacts 56. The wireless transceiver 69 can be implemented using one or more forms of wireless communications, including the IEEE 802.11 computer communications standard, that is Wi-Fi; the 4G mobile phone mobile communications standard; the Bluetooth data exchange standard; or other wireless communications or data exchange standards and protocols. The type of wireless interfacing capability could limit the range of interoperability of the monitor recorder 14; for instance, Bluetooth-based implementations are designed for low power consumption with a short communications range. Use of the wireless transceiver allows real time or near real time offloading of the data recorded by the monitor, including ECG data and button press data. Continuous transfer of data allows faster processing and faster reporting. The data can be transferred using data stitching, such as described in U.S. patent application Ser. No. 17/571,005, filed Jan. 7, 2022 and published as U.S. Patent Pub. 2022/022317 on Jul. 14, 2022, pending, the disclosure of which is hereby incorporated by reference.

The microcontroller 61 includes an expansion port that also utilizes the communications bus. External devices, separately drawing power externally from the battery provided on the electrode patch 15 or other source, can interface to the microcontroller 61 over the expansion port in half duplex mode. For instance, an external physiology sensor can be provided as part of the circuitry 60 of the monitor recorder 14, or can be provided on the electrode patch 15 with communication with the micro-controller 61 provided over one of the electrical contacts 56. The physiology sensor can include an SpO₂ sensor, blood pressure sensor, temperature sensor, respiratory rate sensor, glucose sensor, airflow sensor, volumetric pressure sensing, or other types of sensor or telemetric input sources. For instance, the integration of an airflow sensor is described in commonly-assigned U.S. Pat. No. 9,364,155, entitled “Self-Contained Personal Air Flow Sensing Monitor,” issued Jun. 14, 2016, the disclosure which is incorporated by reference. Despite the presence of the wireless transceiver, flash memory can be important as a backup, such as when a wireless connection is not available.

Finally, the circuitry 60 of the monitor recorder 14 includes patient-interfaceable components, including a tactile feedback button 66, which a patient can press to mark events or to perform other functions, and a buzzer 67, such as a speaker, magnetic resonator or piezoelectric buzzer. The buzzer 67 can be used by the microcontroller 61 to output feedback to a patient such as to confirm power up and initiation of ECG monitoring. Still other components as part of the circuitry 60 of the monitor recorder 14 are possible. For example, the buzzer 67 may be replaced with an LED light to output feedback to a patient.

While the monitor recorder 14 operates under micro control, most of the electrical components of the electrode patch 15 operate passively. FIG. 10 is a functional block diagram showing the circuitry 70 of the extended wear electrode patch 15 of FIG. 4. The circuitry 70 of the electrode patch 15 is electrically coupled with the circuitry 60 of the monitor recorder 14 through an external connector 74. The external connector 74 is terminated through the set of pads 34 provided on the bottom of the non-conductive receptacle 25, which electrically mate to corresponding electrical contacts 56 protruding from the bottom surface of the sealed housing 50 to electrically interface the monitor recorder 14 to the electrode patch 15.

The circuitry 70 of the electrode patch 15 performs three primary functions. First, a battery 71 is provided in a battery compartment formed on the bottom surface of the non-conductive receptacle 25. The battery 71 is electrically interfaced to the circuitry 60 of the monitor recorder 14 as a source of external power. The unique provisioning of the battery 71 on the electrode patch 15 provides several advantages. First, the locating of the battery 71 physically on the electrode patch 15 lowers the center of gravity of the overall wearable monitor 12 and thereby helps to minimize shear forces and the effects of movements of the patient and clothing. Moreover, the housing 50 of the monitor recorder 14 is sealed against moisture and providing power externally avoids having to either periodically open the housing 50 for the battery replacement, which also creates the potential for moisture intrusion and human error, or to recharge the battery, which can potentially take the monitor recorder 14 off line for hours at a time. In addition, the electrode patch 15 is intended to be disposable, while the monitor recorder 14 is a reusable component. Each time that the electrode patch 15 is replaced, a fresh battery is provided for the use of the monitor recorder 14, which enhances ECG monitoring performance quality and duration of use. Finally, the architecture of the monitor recorder 14 is open, in that other physiology sensors or components can be added by virtue of the expansion port of the microcontroller 61. Requiring those additional sensors or components to draw power from a source external to the monitor recorder 14 keeps power considerations independent of the monitor recorder 14. Thus, a battery of higher capacity could be introduced when needed to support the additional sensors or components without effecting the monitor recorders circuitry 60.

Second, the pair of ECG electrodes 38, 39 respectively provided on the distal and proximal ends of the flexible circuit 32 is electrically coupled to the set of pads 34 provided on the bottom of the non-conductive receptacle 25 by way of their respective circuit traces 33, 37. The signal ECG electrode 39 includes a protection circuit 72, which is an inline resistor that protects the patient from excessive leakage current.

Last, in a further embodiment, the circuitry 70 of the electrode patch 15 includes a cryptographic circuit 73 to authenticate an electrode patch 15 for use with a monitor recorder 14. The cryptographic circuit 73 includes a device capable of secure authentication and validation. The cryptographic device 73 ensures that only genuine, non-expired, safe, and authenticated electrode patches 15 are permitted to provide monitoring data to a monitor recorder 14, such as described in commonly-assigned U.S. Pat. No. 9,655,538, entitled “Self-Authenticating Electrocardiography Monitoring Circuit,” issued May 23, 2017, the disclosure which is incorporated by reference.

In a further embodiment, the circuitry 70 of the electrode patch 15 includes a wireless transceiver 75, in lieu of including of the wireless transceiver 69 in the circuitry 60 of the monitor recorder 14, which interfaces with the microcontroller 61 over the microcontroller's expansion port via the external connector 74.

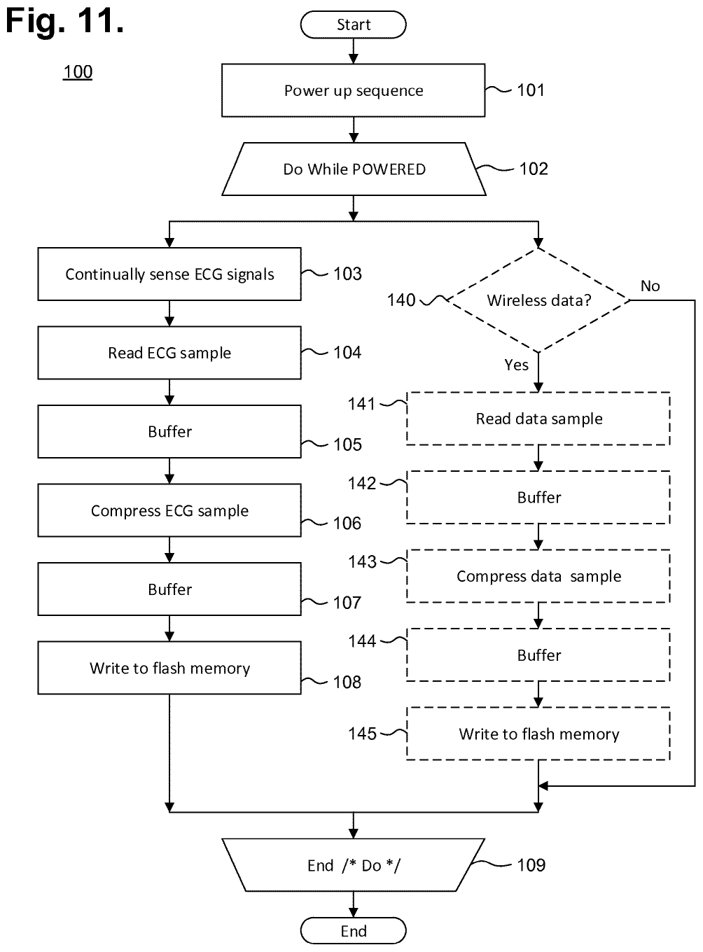

The monitor recorder 14 continuously monitors the patient's heart rate and physiology. FIG. 11 is a flow diagram showing a monitor recorder-implemented method 100 for monitoring ECG data for use in the monitor recorder 14 of FIG. 4. Initially, upon being connected to the set of pads 34 provided with the non-conductive receptacle 25 when the monitor recorder 14 is snapped into place, the microcontroller 61 executes a power up sequence (step 101). During the power up sequence, the voltage of the battery 71 is checked, the state of the flash memory 62 is confirmed, both in terms of operability check and available capacity, and microcontroller operation is diagnostically confirmed. In a further embodiment, an authentication procedure between the microcontroller 61 and the electrode patch 15 are also performed.

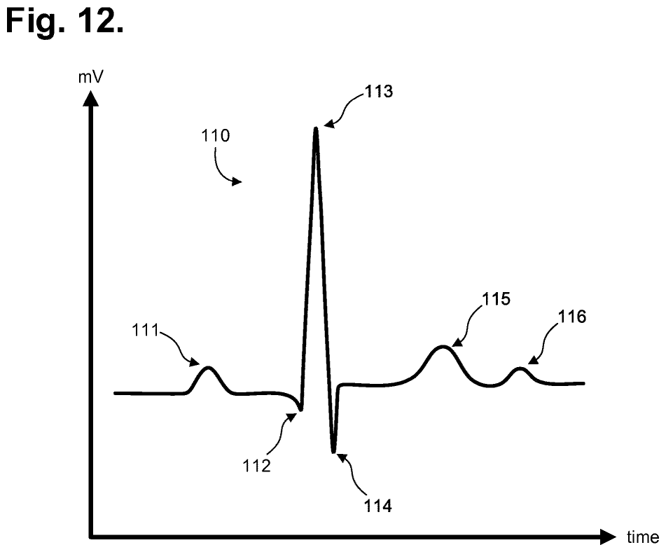

Following satisfactory completion of the power up sequence, an iterative processing loop (steps 102-109) is continually executed by the microcontroller 61. During each iteration (step 102) of the processing loop, the ECG frontend 63 (shown in FIG. 9) continually senses the cutaneous ECG electrical signals (step 103) via the ECG electrodes 38, 29 and is optimized to maintain the integrity of the P-wave. A sample of the ECG signal is read (step 104) by the microcontroller 61 by sampling the analog ECG signal output front end 63. FIG. 12 is a graph showing, by way of example, a typical ECG waveform 110. The x-axis represents time in approximate units of tenths of a second. They-axis represents cutaneous electrical signal strength in approximate units of millivolts. The P-wave 111 has a smooth, normally upward, that is, positive, waveform that indicates atrial depolarization. The QRS complex usually begins with the downward deflection of a Q wave 112, followed by a larger upward deflection of an R-wave 113, and terminated with a downward waveform of the S wave 114, collectively representative of ventricular depolarization. The T wave 115 is normally a modest upward waveform, representative of ventricular depolarization, while the U wave 116, often not directly observable, indicates the recovery period of the Purkinje conduction fibers.

Sampling of the R-to-R interval enables heart rate information derivation. For instance, the R-to-R interval represents the ventricular rate and rhythm, while the P-to-P interval represents the atrial rate and rhythm. Importantly, the PR interval is indicative of atrioventricular (“AV”) conduction time and abnormalities in the PR interval can reveal underlying heart disorders, thus representing another reason why the P-wave quality achievable by the extended wear ambulatory electrocardiography and physiological sensor monitor described herein is medically unique and important. The long-term observation of these ECG indicia, as provided through extended wear of the wearable monitor 12, provides valuable insights to the patient's cardiac function and overall well-being.

Each sampled ECG signal, in quantized and digitized form, is temporarily staged in buffer (step 105), pending compression preparatory to storage in the flash memory 62 (step 106). Following compression, the compressed ECG digitized sample is again buffered (step 107), then written to the flash memory 62 (step 108) using the communications bus. Processing continues (step 109), so long as the monitoring recorder 14 remains connected to the electrode patch 15 (and storage space remains available in the flash memory 62), after which the processing loop is exited and execution terminates. Still other operations and steps are possible.

In a further embodiment, the monitor recorder 14 also continuously receives data from wearable physiology and activity sensors 131 and wearable or mobile communications devices 133 (shown in FIG. 3). The data is received in a conceptually-separate execution thread as part of the iterative processing loop (steps 102-109) continually executed by the microcontroller 61. During each iteration (step 102) of the processing loop, if wireless data is available (step 140), a sample of the wireless is read (step 141) by the microcontroller 61 and, if necessary, converted into a digital signal by the onboard ADC of the microcontroller 61. Each wireless data sample, in quantized and digitized form, is temporarily staged in buffer (step 142), pending compression preparatory to storage in the flash memory 62 (step 143). Following compression, the compressed wireless data sample is again buffered (step 144), then written to the flash memory 62 (step 145) using the communications bus. Processing continues (step 109), so long as the monitoring recorder 14 remains connected to the electrode patch 15 (and storage space remains available in the flash memory 62), after which the processing loop is exited and execution terminates. Still other operations and steps are possible.

The monitor recorder 14 stores ECG data and other information in the flash memory 62 (shown in FIG. 9) using a proprietary format that includes data compression. As a result, data retrieved from a monitor recorder 14 must first be converted into a format suitable for use by third party post-monitoring analysis software. FIG. 13 is a flow diagram showing a method 150 for offloading and converting ECG and other physiological data from an extended wear electrocardiography and physiological sensor monitor 12 in accordance with one embodiment. The method 150 can be implemented in software and execution of the software can be performed on a download station 125, which could be a programmer or other device, or a computer system, including a server 122 or personal computer 129, such as further described supra with reference to FIG. 3, as a series of process or method modules or steps. For convenience, the method 150 will be described in the context of being performed by a personal computer 136 or other connectable computing device (shown in FIG. 3) as middleware that converts ECG data and other information into a format suitable for use by a third-party post-monitoring analysis program. Execution of the method 150 by a computer system would be analogous mutatis mutandis.

Initially, the download station 125 is connected to the monitor recorder 14 (step 151), such as by physically interfacing to a set of terminals 128 on a paired receptacle 127 or by wireless connection, if available. The data stored on the monitor recorder 14, including ECG and physiological monitoring data, other recorded data, and other information are retrieved (step 152) over a hard link 135 using a control program 137 (“Ctl”) or transmitted wirelessly via an application 133 executing on a mobile device 132.

The data retrieved from the monitor recorder 14 is in a proprietary storage format and each datum of recorded ECG monitoring data, as well as any other physiological data or other information, must be converted, so that the data can be used by a third-party post-monitoring analysis program. Each datum of ECG monitoring data is converted by the middleware (steps 153-159) in an iterative processing loop. During each iteration (step 153), the ECG datum is read (step 154) and, if necessary, the gain of the ECG signal is adjusted (step 155) to compensate, for instance, for relocation or replacement of the electrode patch 15 during the monitoring period.

In addition, depending upon the configuration of the wearable monitor 12, other physiological data (or other information), including patient events, such as a fall, peak activity level, sleep detection, detection of patient activity levels and states, and so on, may be recorded along with the ECG monitoring data. For instance, actigraphy data may have been sampled by the actigraphy sensor 64 based on a sensed event occurrence, such as a sudden change in orientation due to the patient taking a fall. In response, the monitor recorder 14 will embed the actigraphy data samples into the stream of data, including ECG monitoring data that is recorded to the flash memory 62 by the micro-controller 61. Post-monitoring, the actigraphy data is temporally matched to the ECG data to provide the proper physiological context to the sensed event occurrence. As a result, the three-axis actigraphy signal is turned into an actionable event occurrence that is provided, through conversion by the middleware, to third party post-monitoring analysis programs, along with the ECG recordings contemporaneous to the event occurrence. Other types of processing of the other physiological data (or other information) are possible.

Thus, during execution of the middleware, any other physiological data (or other information) that has been embedded into the recorded ECG monitoring data is read (step 156) and time-correlated to the time frame of the ECG signals that occurred at the time that the other physiological data (or other information) was noted (step 157). Finally, the ECG datum, signal gain adjusted, if appropriate, and other physiological data, if applicable and as time-correlated, are stored in a format suitable to the backend software (step 158) used in post-monitoring analysis.

In a further embodiment, the other physiological data is embedded within an unused ECG track. For example, the SCP-ENG standard allows multiple ECG channels to be recorded into a single ECG record. The monitor recorder 14, though, only senses one ECG channel. The other physiological data can be stored into an additional ECG channel, which would otherwise be zero-padded or altogether omitted. The backend software would then be able to read the other physiological data in context with the single channel of ECG monitoring data recorded by the monitor recorder 14, provided the backend software implemented changes necessary to interpret the other physiological data. Still other forms of embedding of the other physiological data with formatted ECG monitoring data, or of providing the other physiological data in a separate manner, are possible. Processing continues (step 159) for each remaining ECG datum, after which the processing loop is exited and execution terminates. Still other operations and steps are possible.

Prior to or after data offloading, the data can be processed and analyzed to determine beats and classify segments of beats. The data processing and analysis can occur on the monitor, using an application downloaded on a mobile device of the patient, or at an independent diagnostic facility, as well as a combination of processing on the monitor, via the application and at the facility. For example, the data can be offloaded to the application, which runs beat detection and arrhythmia detection algorithms to help identify data of concern, which can be selected for further review by a human reviewer at a diagnostic review center. In a further embodiment, beat detection can occur on the device, while arrhythmia detection can occur via the application, and manual review is performed at a diagnostic facility to review data identified by the arrhythmia detection.

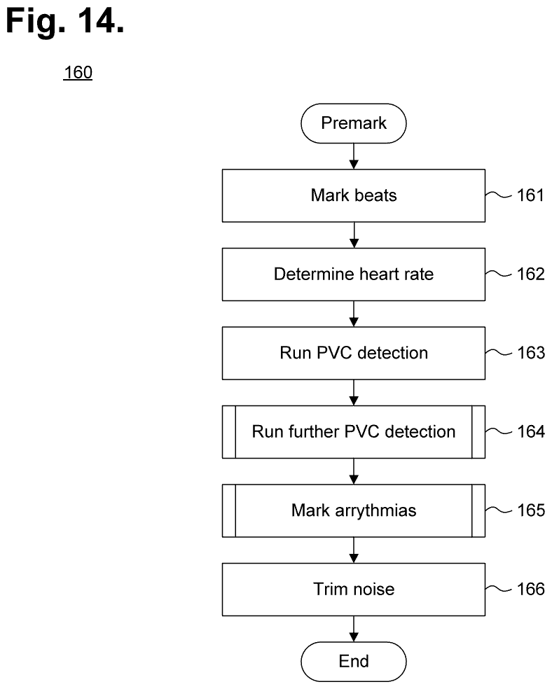

Rhythms of heart beats are classified to premark possible arrhythmia for an ECG reviewer to focus on for further review and classification. FIG. 14 is a flow diagram showing, by way of example, a method 160 for beat-based arrhythmia detection. ECG data recorded by the monitor is processed using a beat detection algorithm to mark (step 161) occurrences of heart beats. Various types and quantities of beat detection algorithms can be used to mark the occurrences of heat beats. For example, a first beat detection algorithm and a second beat detection algorithm can both be used to determine which occurrences of heat beats should be marked. Such results may be compared for increased accuracy. The Veritas ECG interpretation algorithm is one example of a beat detection algorithm that may be used in step 161. After marking the occurrences of heat beats, a distance between consecutive beats is calculated to determine (step 162) a heart rate of the patient. It should be appreciated that determining the heart rate (step 162) may occur at many points in the flow diagram of FIG. 14. Using the heart rate data, premature ventricular contraction (PVC) detection can be performed (step 163). In one embodiment, PVC detection can occur by establishing an average heart rate of the patient by computing all beat times over narrow windows of beat data. Individual heart beats are then compared with the average and any individual heart beats that occur earlier than the average are determined to be premature. After PVC detection is run, beats are spit into PVC marked beats and normal beats.

A second round of PVC detection can occur (step 164) to identify any previously PVC beats that were misclassified. Ventricular tachycardia is a series of PVCs, so being able to correctly identify more PVCs simplifies the challenge of finding ventricular tachycardia events because the entire detection algorithm doesn't need to be reinvented since data from the first round, such as beats identified as PVC, is used as a starting point.

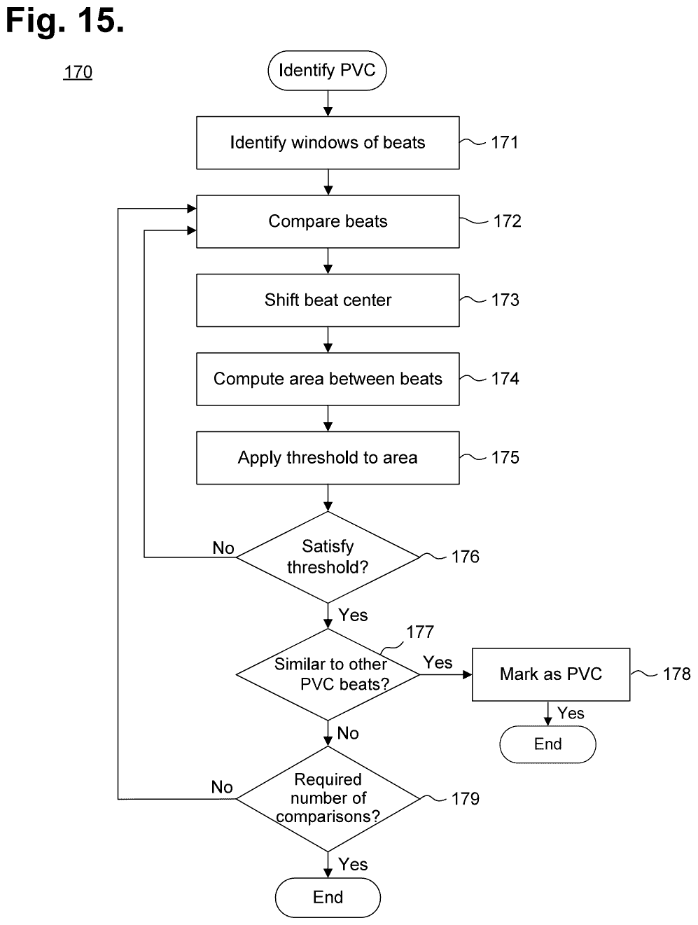

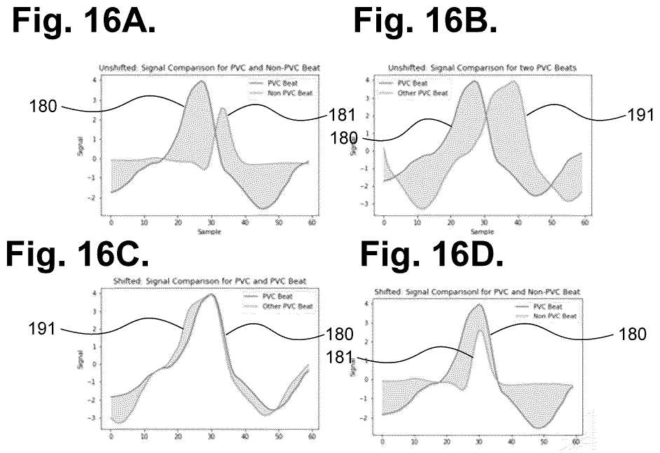

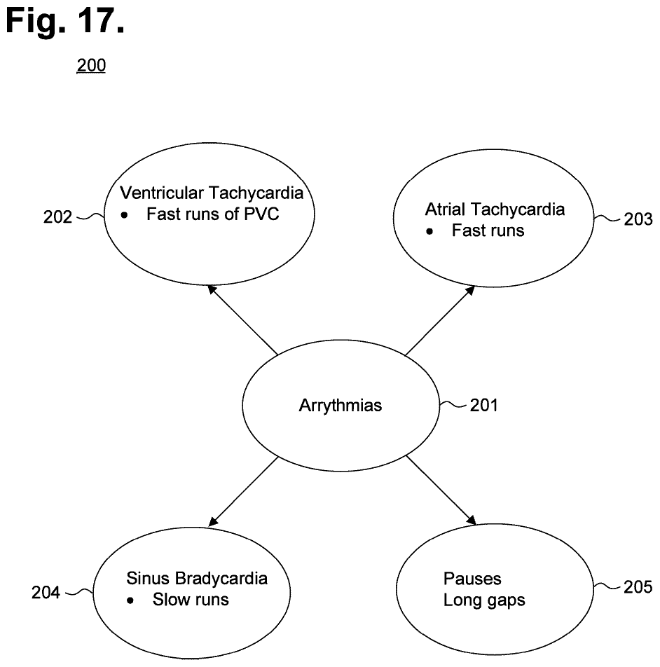

The second round of PVC is performed by plotting the identified PVC beats with nearby beats and detecting additional PVC beats based on a similar morphology with the already identified PVC beats, as further described in detail below with respect to FIGS. 15 and 16. After, runs of beats can be classified or marked (step 165) as representing one or more cardiac conditions. For example, fast runs of PVC beats can be classified as ventricular tachycardia. Arrhythmia marking is further described below with respect to FIG. 17. Finally, a noise detection algorithm can be run on the cardiac data and any noise detected can be trimmed (step 166). For example, when a window or segment of beats is more than 80% noise, the noise can be trimmed. In one embodiment, regions of data and noise are differentiated by training an Artificial Intelligence system to recognize the difference and a location of the noise detected.

Running a second round of PVC detection can be helpful to correct any misclassified PVC beats by the first round of PVC detection. For example, during the first round of detection, several PVC beats can be misclassified as non-PVC beats if there are multiple fast beats in a sequence, as compared to a single beat that is fast and in between two average beats. FIG. 15 is a flow diagram showing, by way of example, a method 170 for identifying PVC beats. At least one window of beats is identified (step 171) for PVC detection. In one embodiment, each window can include 100 to 200 beats, as well as other numbers of beats. Within the window, each nearby non-PVC beat or at least a portion of the nearby non-PVC beats in the window are compared (step 172) with a PVC beat identified during the first round of detection. In one embodiment, each of the windows can be generated by first identifying a PVC beat from the first round and then selecting the 100-200 closest beats to the identified PVC beat based on time and/or proximity to the identified PVC beat. A size of the windows can be determined based on a size of the beat recording. For example, if a recording has fewer than 1000 beats, each identified PVC beat is compared to all 1000 beats. However, it there are more than 1000 beats, a smaller window size can be used, such as 500 beats.

The identified PVC beat can be compared with each beat in the window or a portion of the beats by lining the PVC and non-PVC beats on top of one another to determine a similarity of the beat shapes. In one embodiment, the beats can be visually displayed via a graph for comparison; however, the beats can also be compared without the use of a graph, such as a by interpreting the signals as a series of numbers. When used for visual purposes, the graph can include sample periods along an x-axis and signal along a y-axis. The time is measured a single voltage sample recorded by a cardiac device. In one example, there can be 512 samples every three seconds; however, other samples and time periods are possible. The signal along the y-axis represents a voltage of the ECG signal measured by the cardiac device. For comparison, the center of the PVC beat and nearby beat can be shifted (step 173) to improve alignment of the beats. Once adjusted, an area between the PVC beat and nearby beat is calculated (step 174).

A threshold can be applied (step 176) to the calculated area to determine whether the nearby beat is similar to the PVC beat. The similarity is quantified by the difference represented by the area calculated between the PVC beat and the nearby beat. In one example, a threshold difference can be 20% and if the area is equal to or less than 20%, then the nearby beat is determined to be similar to the PVC beat. If the area difference does not satisfy the threshold (step 176), the nearby non-PV beat is determined to be dissimilar to the PVC beat. If the area between the beats satisfies the threshold, the nearby beat can be determined to be similar to the PVC beat and can be classified as a PVC beat. However, in a further embodiment, the nearby beat must be similar to two or more PVC beats before the nearby beat is determined to be a PVC beat. For example, a further determination (step 177) is made as to whether the nearby beat is similar to at least one further PVC beat identified by the first round of PVC detection. If yes, the nearby beat is marked as representing a PVC beat. However, if the nearby beat is not similar (step 177) to another PVC beat, the nearby beat is compared with further PVC beats (step 172) until the nearby beat is compared (step 179) with a sufficient or required number of PVC beats. Also, if a sufficient number of comparisons have not been made (step 179), including all the beats in the window, the non-PVC beat is compared (step 172) with further beats in the window.

The similarity of the PVC and non-PVC beats is determined using a difference determined between graphed representations of the beats. FIGS. 16A-D each represent a graph of a PVC beat and non-PVC beat. FIG. 16A is a block diagram showing, by way of example, a graph of a PVC beat 180 and a non-PVC beat 181. The beats are mapped along an x-axis that represents sample duration and a y-axis that represents signal measure and are not aligned using a particular point. FIG. 16B is a block diagram showing, by way of example, graph of a PVC beat 180 and a different non-PVC beat 191. The beats are graphed solely on the sample duration and are not aligned using a particular point.

To more easily quantify a difference of the beats using an area between the graphed representations, the PVC and non-PVC beats are aligned. FIG. 16C is a block diagram showing, by way of example, graph of a PVC beat 180 and the non PVC beat 191 of FIG. 16B. The PVC and non-PVC beats are aligned along the x-axis such that the highest points of each beat are aligned. Specifically, the beats are aligned to appear most similar to one another, including for example matching peaks and valleys of the beats or matching beginning and end times of the beats. The area between the aligned beats is determined and represents an amount of difference between the beats, which is used in turn to determine a measure of similarity of the beats. A threshold is applied to the area and a determination is made as to whether the non-PVC beat is so similar to the PVC beat that the non-PVC beat should be classified as PVC.

FIG. 16D is a block diagram showing, by way of example, graph of a PVC beat 180 and the non-PVC beat 181 of FIG. 16A. The PVC and non-PVC beats are aligned using the highest point of each beat such that the highest point of the beats are centered along the x-axis. The area between the graphed beats is determined and represents an amount of difference between the beats, which is used to represent a measure of similarity of the beats. The area between the PVC beat 180 and non-PVC beat 181 is more than the area between the PVC beat 180 and non-PVC beat 191 of FIG. 16C and may be too much of a difference to consider changing the marking of the non-PVC beat to a PVC beat, depending on the threshold.

The marked PVC beats and the ECG data, are utilized by an arrhythmia detector, which can be run via an application on a mobile device of a patient or a program run in a cloud computing environment. The arrhythmia detector can mark segments of the ECG data as representing a cardiac condition. In one embodiment, such marking can be an automatic diagnosis or alternatively, the marking is used to flag that segment of ECG data for further review and processing. Regardless, such segment of ECG data can be selected and placed into a report and/or patient record.

In one embodiment, the arrhythmia detector is a non-AI algorithm for detecting atrial tachycardia (“AT”), ventricular tachycardia (“VT”), pauses and low heart rate to look for critical events that need to be reviewed by a human. The algorithm can use the placement of beats provided by the beat detection and the beat category provided by the two rounds of PVC detection to make classifications. Medical professionals, such as ECG technicians can set duration and heart rate thresholds which the detection algorithm uses to determine which events to mark. The duration and heart rate thresholds allow the selection of a subset of arrhythmias to be identified in a patient. Duration refers to the number of beats in an arrhythmia for atrial tachycardia and ventricular tachycardia and seconds of time where the patient presented the arrhythmia for pauses and sinus bradycardia. Meanwhile, the heart rate thresholds can include a particular window or threshold for heart rate. The subset of arrhythmias can be selected by an individual, such as a medical professional or automatically. In a clinical sense, selection of the subset by a medical professional allows, doctors and nurses to specify what they consider to be “severe.” For example, 200 beats per minute isn't concerning for a 15 year old, but is very concerning for a 90 year old.

Using the ECG data, PVC classifications, heart rate and duration, occurrences of different cardiac conditions can be identified. FIG. 17 is a block diagram 200 showing, by way of example, different arrhythmia conditions 201. Ventricular tachycardia 202 can be identified based on fast runs of PVC beats by using heart rate regularity to identify skipped beats and ignoring runs with extremely irregular heart rates. Fast runs of PVCs are a series of PVC beats with a heart rate above or around the heart rate threshold. In one embodiment, the runs or series can include three or more beats; however, other numbers of beats are possible. Heart rate regularity helps identify places where a beat detector, performing beat detection, has made a mistake. For example, when the heart rate suddenly halves for one beat and then returns to a normal heart rate, we can assume that the beat detector missed a beat.

Ventricular tachycardia 202 can also be identified and marked based on long gaps between beats, which indicate that a beat has been missed by the beat detector. In one example, the long gap can be determined by a threshold amount of time and if the long gap exceeds the threshold time, ventricular tachycardia can be identified. Atrial tachycardia can be identified based on a fast run of beats and can require, for example, that an average heart rate of the fastest 80% of beats to exceed heart rate threshold. Runs with an extremely irregular heart rate can be ignored. Also, sinus bradycardia 204 can be identified based on a slow run of beats and can require, for example, that the average heart rate of the slowest 90% of beats be below a heart rate threshold. Other percentages and threshold values are possible. Further, pauses and long gaps 205 in heart rate, such as ventricular contraction, can also be detected. The marked beat segments can be provided in a report for providing to a reviewer or physician for further review or diagnosis. Additionally, the report can be stored in the patient's medical record.

Although the above description focuses on processing and arrhythmia detection using data recorded by a cutaneous monitor placed on the skin of a patient, data from implantable cardiac devices can also be used.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for beat-based arrhythmia detection, comprising:
a database to store cardiac data;
a cardiac monitor to continuously transmit the cardiac data collected to the database via a mobile application running on a mobile device associated with a patient; and
a server comprising a central processing unit, memory, an input port, and an output port, wherein the central processing unit is configured to:
identify beats from the cardiac data;
run detection for premature ventricular contractions and identify one or more beats as representing premature ventricular contractions;
compare at least one of the identified beats with each of a plurality of nearby beats by lining up the nearby beat and the identified beat;
determine an area between the identified beat and each nearby beat; and
mark at least one of the nearby beats as representing premature ventricular contraction when the area between that nearby beat and the identified beat satisfies a threshold level of similarity.

2. The system for beat-based arrhythmia detection according to claim 1, wherein the cardiac data is transmitted from the cardiac monitor using mobile cardiac telemetry.

3. The system for beat-based arrhythmia detection according to claim 1, wherein the cardiac monitor comprises a cutaneous monitor placed on skin of the patient or an implantable monitor.

4. The system for beat-based arrhythmia detection according to claim 1, wherein the cardiac data is transmitted from the cardiac monitor via a wireless transceiver.

5. The system for beat-based arrhythmia detection according to claim 4, wherein the wireless transceiver comprises a Bluetooth chip.

6. The system for beat-based arrhythmia detection according to claim 1, wherein the cardiac monitor further comprises an electrocardiography patch, an externally-powered micro-controller, and an electrocardiographic front end circuit electrically interfaced to the micro-controller and operable to sense electrocardiographic signals as the cardiac data.

7. The system for beat-based arrhythmia detection according to claim 1, wherein the nearby beat and the identified beat are lined up based on one of start time and stop time, and shape.

8. The system for beat-based arrhythmia detection according to claim 1, wherein the detection for premature ventricular contractions is performed by establishing an average heart rate of the patient by computing all beat times for heart beats over narrow windows of heart beat data, comparing individual heart beats with the average heart rate, and identifying any individual heart beats that occur earlier than the average as premature.

9. The system for beat-based arrhythmia detection according to claim 1, wherein the central processing unit performs arrhythmia detection on the premature ventricular contraction beats and the cardiac data.

10. The system for beat-based arrhythmia detection according to claim 9, wherein the central processing unit flags one or more segments of the cardiac data as representing a cardiac condition based on the arrhythmia detection.

11. A method for beat-based arrhythmia detection, comprising:
continuously transmitting cardiac data from a cardiac monitor worn by a patient to storage via a mobile application running on a mobile device associated with the patient;
identifying beats from the cardiac data;
running detection for premature ventricular contractions and identifying one or more beats as representing premature ventricular contractions;
comparing at least one of the identified beats with each of a plurality of nearby beats by lining up the nearby beat and the identified beat;
determining an area between the identified beat and each nearby beat; and
marking at least one of the nearby beats as representing premature ventricular contraction when the area between that nearby beat and the identified beat satisfies a threshold level of similarity.

12. The method for beat-based arrhythmia detection according to claim 11, wherein the cardiac data is transmitted from the cardiac monitor using mobile cardiac telemetry.

13. The method for beat-based arrhythmia detection according to claim 11, wherein the cardiac monitor comprises a cutaneous monitor placed on skin of the patient or an implantable monitor.

14. The method for beat-based arrhythmia detection according to claim 11, further comprising:
transmitting the cardiac data from the cardiac monitor via a wireless transceiver.

15. The method for beat-based arrhythmia detection according to claim 14, wherein the wireless transceiver comprises a Bluetooth chip.

16. The method for beat-based arrhythmia detection according to claim 11, wherein the cardiac monitor further comprises an electrocardiography patch, an externally-powered micro-controller, and an electrocardiographic front end circuit electrically interfaced to the micro-controller and operable to sense electrocardiographic signals as the cardiac data.

17. The method for beat-based arrhythmia detection according to claim 11, wherein the nearby beat and the identified beat are lined up based on one of start time and stop time, and curves.

18. The method for beat-based arrhythmia detection according to claim 11, further comprising:
performing the detection for premature ventricular contractions, comprising:
establishing an average heart rate of the patient by computing all beat times for heart beats over narrow windows of heart beat data; and
comparing individual heart beats with the average heart rate; and
identifying any individual heart beats that occur earlier than the average as premature.

19. The method for beat-based arrhythmia detection according to claim 11, further comprising:
performing arrhythmia detection on the premature ventricular contraction beats and the cardiac data.

20. The method for beat-based arrhythmia detection according to claim 19, further comprising:
flagging one or more segments of the cardiac data as representing a cardiac condition based on the arrhythmia detection.
